(19) United States
(12) Patent Application Publication
(10) Pub. No.: US 2021/0188631 A1
(43) Pub. Date: Jun. 24, 2021

(54) CARBON MEDIATED WATER-SPLITTING USING FORMALDEHYDE

(71) Applicant: SABIC Global Technologies B.V., Bergen op Zoom (NL)

(72) Inventors: Khalid Albahily, Thuwal (SA); Balamurugan Vidjayacoumar, Thuwal (SA); Sandro Gambarotta, Ottawa (CA); Nicholas P. Alderman, Ottawa (CA)

(21) Appl. No.: 16/076,116

(22) PCT Filed: Feb. 16, 2017
(86) PCT No.: PCT/IB2017/050883

§ 371 Date: Aug. 7, 2018

Related U.S. Application Data
(60) Provisional application No. 62/300,175, filed on Feb. 26, 2016.

(51) Int. Cl. C01B 3/26; C07C 45/41; C07C 51/295

(52) U.S. Cl. CPC C01B 3/26; C07C 47/04; C07C 51/295; C07C 45/41

(57) ABSTRACT

Methods of producing hydrogen are described. A method can include combining an aqueous base, formaldehyde, and a transition metal complex having a coordination bond between a transition metal and a leaving group to form a homogeneous aqueous solution having a basic pH. The leaving group dissociates from the transition metal complex in response to light and/or the basic pH of the solution to produce hydrogen (H2) gas and formate or a salt thereof from the formaldehyde present in the homogeneous aqueous solution. Subsequent hydrogenation of the formate or a salt thereof produces formaldehyde.

### CROSS REFERENCE TO RELATED APPLICATIONS

This application claims the benefit of priority of U.S. Provisional Patent Application No. 62/300,175 filed Feb. 26, 2016, which is hereby incorporated by reference in its entirety.

### BACKGROUND OF THE INVENTION

### A. Field of the Invention

The invention generally concerns the production of hydrogen and formate from an aqueous solution that includes formaldehyde.

### B. Description of Related Art

There is increasing global demand for hydrogen gas. Conventional technology produces hydrogen from steam reforming of methane as shown in the equations (1) and (2) below. The major source of the methane is from natural gas:

CH₄+H₂O→CO+3H₂ (1)

CO+H₂O CO₂+H₂ (2)

Due to the depletion of fossil fuels, there is a necessity to find an alternative feedstock to meet the growing demand for hydrogen production globally.

Alternative processes for hydrogen production have been proposed (for example, water-splitting, thermal dehydrogenation of formic acid, catalytic dehydrogenation of small organic molecules, electrolysis of water/formaldehyde solutions, thermal dehydrogenation of amino-boranes and the like). Dehydrogenation of small organic molecules such as formic acid, methanol and formaldehyde has been attempted. Dehydrogenation of formic acid into hydrogen and carbon dioxide suffers in that the reaction is inefficient as formic acid has a low hydrogen content (about 4.4 wt. %). Further, the production of carbon dioxide can be problematic.

There have been attempts to use formaldehyde as an additive in water-splitting reactions to produce hydrogen and oxygen. Many of these processes can require additional materials and/or use high temperatures, thereby making the processes inefficient and difficult to scale-up for mass hydrogen gas production. By way of example, U.S. Pat. No. 4,175,013 to Barnert et al. describes the electrolysis of water using formaldehyde as an acidic electrolyte to produce oxygen and a low molecular weight hydrocarbon International Application Publication No. WO 2015/003680 to Prechtl et al. describes thermal process for generating hydrogen by heating formaldehyde-containing water at 95° C. in the presence of a catalyst having a dimeric form of ruthenium with aromatic hydrocarbon ligands. Kumar et al. in “Role of sodium hydroxide for hydrogen gas production and storage”, Materials and processes for energy: communicating current research and technological developments, (A. Mendez-Filas, Ed.), 2013, p. 258, describes that concentrated sodium hydroxide (19M) is required to produce significant amounts of hydrogen from formaldehyde.

In addition to the inefficiencies of the systems discussed above, photocatalytic attempts to produce hydrogen from aqueous formaldehyde solutions have typically relied on water splitting to generate electron holes that oxidize the formaldehyde to formic acid. Subsequent photo-oxidation of the formic acid produces hydrogen and carbon dioxide through a multi-step process shown in equations (3) through (9) below. By way of example, Ni et al. in a review of photocatalytic water splitting using TiO₂ for hydrogen production discloses the use of formaldehyde as an electron donor for photocatalytic hydrogen production (See, Renewable & Sustainable Energy Review, 2007, 11, 401-425). The use of platinum doped TiO₂ photocatalysts to generate hydrogen from formaldehyde under photocatalytic conditions has been described by Chowdhury et al. in “Sacrificial Hydrogen Generation from Formaldehyde with Pt/TiO₂ Photocatalyst in Solar Radiation,” Ind. Eng. Chem. Res., 2013, Vol. 52(14), pp. 5023-5029 and Li et al., “Photocatalytic production of hydrogen in single component and mixture systems of electron donors and monitoring adsorption of donors by in situ infrared spectroscopy”, Chemosphere, 2003, Vol. 53 (5), 843-850.

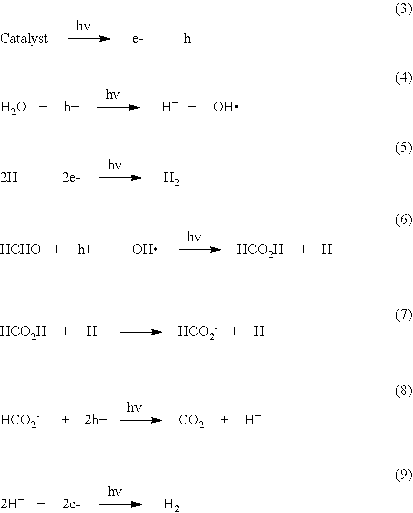

### SUMMARY OF THE INVENTION

A discovery has been made that provides a solution to the aforementioned problems and inefficiencies associated with the generation of hydrogen from small organic molecules such as formaldehyde. The discovery is premised on the use of a homogenous aqueous solution having a basic pH that can be used to produce hydrogen and formate. In particular, the aqueous solution can have formaldehyde (e.g., para-formaldehyde) and a transition metal complex having a coordination bond between a transition metal and a leaving group that dissociates from the transition metal complex in response to light (e.g., iron containing photocatalyst) and/or the basic pH of the solution. The formaldehyde and the transition metal complex can be solubilized in the aqueous solution. Hydrogen (H₂) gas and formate anions can be produced directly from the formaldehyde. Addition of a second catalyst (e.g., a photocatalyst) to the solution can then catalyze the hydrogenation of the produced formate anions with water to produce formaldehyde (e.g., methanediol) and oxygen. The produced formaldehyde can be recycled/reused to produce more H₂ gas and formate anions. Notably, this cyclical process can be performed in the aforementioned aqueous solution and operated at room temperature conditions (e.g., the aqueous solution can have a temperature of 15° C. to 30° C., or more preferable 20° C. to 30° C., or even more preferably 20° C. to 25° C. In some embodiments, due to the cyclic nature of the reaction, formate or a salt thereof can be used as the starting material to generate formaldehyde in situ. Additionally, this system is oxygen-resilient, chemically robust, and energy efficient, thereby allowing for large scale hydrogen production to meet the ever increasing hydrogen gas demands of the chemical and petrochemical industries. Even further, the system of the present invention can avoid the costs associated with conventional photocatalysts that contain expensive noble metals (e.g., Pd/TiO₂) and can limit or avoid the production of by-products such as carbon dioxide. Without wishing to be bound by theory, it is believed that enhanced efficiency of the system is due to the fact that the H₂ evolution occurs in the homogeneous phase of the aqueous solution combined with the recycling of produced formate to formaldehyde. Notably, by-products such as carbon monoxide, carbon dioxide, methanol, methane, and/or oxygen gas (O₂) are not produced in this system.

In a certain aspect of the invention, methods of producing hydrogen are described. A method can include (a) combining an aqueous base, formaldehyde, and a transition metal complex having a coordination bond between a transition metal and a leaving group to form a homogeneous aqueous solution having a basic pH, (b) producing hydrogen (H₂) gas and formate or a salt thereof from the formaldehyde present in the homogeneous aqueous solution and (c) hydrogenating the formate or a salt thereof to produce formaldehyde. The leaving group can dissociate from the transition metal complex in response to light and/or the basic pH of the solution. Oxygen gas (O₂) can also be produced in step (c). All, or a portion thereof, of the formaldehyde produced in step (c) can be recycled or used in steps (a) and/or (b). The formate or salt thereof can be hydrogenated with hydrogen obtained from the water comprised in the homogenous aqueous solution. A heterogeneous catalyst (e.g., a catalyst that is partially or fully suspended in the aqueous solution) capable of catalyzing the hydrogenation of formate a salt thereof can be used in step (c). The catalyst can be a metal oxide photocatalyst (for example, Bi₂WO₆, BiVO₄, LaCoO₃, CuWO₄, BiCu₂VO₆, Au/TiO₂, Cr₂WO₆, or combinations thereof). In some embodiments, steps (a) through (c) are done in one reaction zone under photo-catalytic conditions. In other embodiments, one or more reaction zones can be used (e.g., a thermal or dark zone and a photocatalytic zone). Conditions for the reactions in steps (a), (b) or (c) can include a temperature from greater than 0° C. to less than 50° C., preferably from 10° C. to 40° C., more preferably from 15° C. to 30° C., and most preferably from 20° C. to 25° C. The formaldehyde and the transition metal complex can be fully solubilized in the homogeneous aqueous solution. Without wishing to be bound by theory, it is believed that the leaving group of the transition metal complex can be replaced by a hydroxyl ion to form a transition metal-hydroxyl coordination bond. The transition metal-hydroxyl coordination bond can then react with the formaldehyde to produce the hydrogen and formate or salt thereof. Transition metals can include iron (Fe), ruthenium (Ru), iridium (Ir), copper (Cu), or silver (Ag), which result in an Fe complex, preferably an Fe(II) complex, a Ru complex, preferably a Ru(III) complex, an Ir complex, preferably an Ir(III) complex, a Cu complex, preferably a Cu(I) complex, or a Ag complex, preferably an Ag(I) complex. Dissociation of the leaving group from the transition metal complex can be induced by applying light to the solution or changing the pH of the solution. In a particular embodiment, the transition metal complex can be ferricyanide (Fe(CN)₆)⁴⁻) or a salt thereof and at least one of the (CN⁻) groups dissociates from the ferricyanide in response to sunlight and/or artificial light (e.g., a xenon lamp, a fluorescent light, an LED light, an incandescent light, an ultraviolet (UV) light, or any combination thereof). In another embodiment, the transition metal complex includes a halide (fluoride (F⁻), chloride (Cl⁻), bromide (Br⁻), iodide (I⁻), or astatide (At⁻), preferably Cl⁻) leaving group. The halide can dissociate from the transition metal complex in response to a change in pH. In some instances, a molar ratio of formaldehyde to base can be equal to or less than 2:1, preferably equal to or less than 1.5:1, more preferably equal to or less than 1.2:1, even more preferably from 0.5:1 to 1.5:1, or most preferably from 1:1 to 1.3:1. Formaldehyde can be para-formaldehyde, hydrated formaldehyde, or a combination thereof and/or produced from methanol (e.g., from the oxidation of methanol reaction). An alkali base (e.g., NaOH) can be used to adjust the pH of the homogeneous aqueous solution to a pH from 8 to 14, preferably 10 to 14, and most preferably 12 to 14. In the present invention, an external bias is not used to produce the hydrogen and formate or salt thereof.

In another aspect of the present invention, a homogeneous aqueous solution having a basic pH and being capable of producing hydrogen (H₂) gas and formate or a salt thereof is described. The composition can include an aqueous base, formaldehyde, and a transition metal complex having a coordination bond between a transition metal and a leaving group. The aqueous base, formaldehyde and transition metal complex form a homogeneous mixture with the aqueous base, formaldehyde and transition metal complex being partially or completely solubilized in the mixture. As previously discussed, the leaving group can dissociate from the transition metal complex in response to light and/or the basic pH of the solution, and the water comprised in the homogeneous aqueous solution is capable of hydrogenating the produced formate or salt thereof. A heterogeneous catalyst (e.g., a catalyst that is partially or fully suspended in the aqueous solution) describe above can be used to catalyze the hydrogenation of the produced formate or salt thereof

In yet another aspect of the present invention, a system for producing hydrogen (H₂) gas and formate or a salt thereof from formaldehyde is described. The system can include (a) a container that includes the homogeneous aqueous solution of described above or throughout the specification; and (b) optionally, a light source for illuminating the aqueous solution. The light source can be sunlight and/or an artificial light source (e.g., a xenon lamp, a florescent light, an LED light, an incandescent light, an ultraviolet (UV) light, or any combination thereof). One or more portions of the container can be transparent and/or opaque. In a particular aspect, the system does not include an external bias to produce hydrogen or formate or a salt thereof.

In the context of the present invention 59 embodiments are described. Embodiment 1 includes a method of producing hydrogen that includes combining an aqueous base, formaldehyde, and a transition metal complex having a coordination bond between a transition metal and a leaving group to form a homogeneous aqueous solution having a basic pH, wherein the leaving group dissociates from the transition metal complex in response to light and/or the basic pH of the solution; producing hydrogen (H₂) gas and formate or a salt thereof from the formaldehyde present in the homogeneous aqueous solution; and hydrogenating the formate or a salt thereof to produce formaldehyde. Embodiment 2 is the method of embodiment 1, wherein the formate or salt thereof is hydrogenated with hydrogen obtained from water comprised in the homogenous aqueous solution. Embodiment 3 is the method of embodiment 2, wherein a heterogeneous catalyst catalyzes the hydrogenation of formate or a salt thereof with water reaction. Embodiment 4 is the method of embodiment 3, wherein the heterogeneous catalyst is not partially solubilized or not fully solubilized in the homogenous aqueous solution. Embodiment 5 is the method of embodiment 4, wherein the heterogeneous catalyst is a metal oxide photocatalyst selected from Bi₂WO₆, BiVO₄, LaCoO₃, CuWO₄, BiCu₂VO₆, Au/TiO₂, Cr₂WO₆, or combinations thereof. Embodiment 6 is the method of any one of embodiments 1 to 5, wherein the formaldehyde produced from step (c) is recycled/used in steps (a) and/or (b). Embodiment 7 is the method of any one of embodiments 1 to 6, wherein oxygen gas (O₂) is produced in step (c). Embodiment 8 is the method of any one of embodiments 1 to 7, wherein steps (a), (b), and/or (c) are each performed at a temperature from greater than 0° C. to less than 50° C., preferably from 10° C. to 40° C., more preferably from 15° C. to 30° C., and most preferably from 20° C. to 25° C. Embodiment 9 is the method of any one of embodiments 1 to 8, wherein a hydroxide ion replaces the leaving group to form a transition metal-hydroxyl coordination bond, and wherein the transition metal complex having the transition metal-hydroxyl coordination bond reacts with the formaldehyde to produce the hydrogen and formate or salt thereof. Embodiment 10 is the method of any one of embodiments 1 to 9, wherein the transition metal is iron (Fe), ruthenium (Ru), iridium (Ir), copper (Cu), or silver (Ag). Embodiment 11 is the method of embodiment 10, wherein the transition metal complex is an Fe complex, preferably an Fe(II) complex. Embodiment 12 is the method of embodiment 11, wherein the transition metal complex is a Ru complex, preferably a Ru(III) complex. Embodiment 13 is the method of embodiment 11, wherein the transition metal complex is an Ir complex, preferably an Ir(III) complex. Embodiment 14 is the method of embodiment 11, wherein the transition metal complex is a Cu complex, preferably a Cu(I) complex. Embodiment 15 is the method of embodiment 11, wherein the transition metal complex is a Ag complex, preferably an Ag(I) complex. Embodiment 16 is the method of any one of embodiments 1 to 15, wherein the leaving group dissociates from the transition metal complex in response to light. Embodiment 17 is the method of embodiment 16, wherein the leaving group is a cyano group (CN⁻). Embodiment 18 is the method of embodiment 17, wherein the transition metal complex is ferricyanide (Fe(CN)₆)⁴⁻) or a salt thereof. Embodiment 19 is the method of any one of embodiments 17 to 18, wherein the light is sunlight or artificial light, or a combination thereof. Embodiment 20 is the method of embodiment 19, wherein the artificial light is from a xenon lamp, a fluorescent light, an LED light, an incandescent light, an ultraviolet (UV) light, or any combination thereof. Embodiment 21 is the method of any one of embodiments 1 to 20, wherein the leaving group dissociates from the transition metal in response to the basic pH of the solution. Embodiment 22 is the method of embodiment 21, wherein the leaving group is a halide. Embodiment 23 is the method of embodiment 22, wherein the halide is fluoride (F⁻), chloride (Cl⁻), bromide (Br⁻), iodide (I⁻), or astatide (At⁻), preferably Cl⁻. Embodiment 24 is the method of any one of embodiments 1 to 23, wherein the molar ratio of formaldehyde to base is equal to or less than 2:1, preferably equal to or less than 1.5:1, more preferably equal to or less than 1.2:1, even more preferably from 0.5:1 to 1.5:1, or most preferably from 1:1 to 1.3:1. Embodiment 25 is the method of any one of embodiments 1 to 24, wherein the formaldehyde is para-formaldehyde, hydrated formaldehyde, or a combination thereof. Embodiment 26 is the method of any one of embodiments 1 to 21, wherein the formaldehyde is produced from methanol by oxidation of the methanol. Embodiment 27 is the method of any one of embodiments 1 to 26, wherein the base is NaOH. Embodiment 28 is the method of any one of embodiments 1 to 27, wherein the homogeneous aqueous solution has a pH from 8 to 14, preferably 10 to 14, and most preferably 12 to 14. Embodiment 29 is the method of any one of embodiments 1 to 28, wherein an external bias is not used to produce the hydrogen and formate or salt thereof. Embodiment 30 is the method of any one of embodiments 1 to 29, wherein the formaldehyde and the transition metal complex are fully solubilized in the homogeneous aqueous solution. Embodiment 31 is the method of any one of embodiments 1 to 30, wherein by-products such as carbon monoxide, carbon dioxide, methanol, methane, and/or oxygen gas (O₂) are not produced in the method.

Embodiment 32 is a homogeneous aqueous solution having a basic pH and being capable of producing hydrogen (H₂) gas and formate or a salt thereof. The composition of embodiment 32 includes an aqueous base, formaldehyde, and a transition metal complex having a coordination bond between a transition metal and a leaving group, wherein the leaving group dissociates from the transition metal complex in response to light and/or the basic pH of the solution, and wherein water comprised in the homogeneous aqueous solution is capable of hydrogenating the formate or salt thereof. Embodiment 33 is the homogeneous aqueous solution of embodiment 32, further comprising a heterogeneous catalyst that catalyzes the hydrogenation of formate or a salt thereof from water. Embodiment 34 is the homogenous aqueous solution of embodiment 33, wherein the catalyst is a photocatalyst selected from Bi₂WO₆, BiVO₄, LaCoO₃, CuWO₄, BiCu₂VO₆, Au/TiO₂, Cr₂WO₆, or any combination thereof. Embodiment 35 is the homogeneous aqueous solution of any one of embodiments 32 to 34, wherein the transition metal complex metal is iron (Fe), ruthenium (Ru), iridium (Ir), copper (Cu), or silver (Ag). Embodiment 36 is the homogeneous aqueous solution of embodiment 35, wherein the transition metal complex is an Fe complex, preferably an Fe(II) complex. Embodiment 37 is the homogeneous aqueous solution of embodiment 35, wherein the transition metal complex is a Ru complex, preferably a Ru(III) complex. Embodiment 38 is the homogeneous aqueous solution of embodiment 35, wherein the transition metal complex is an Ir complex, preferably an Ir(III) complex. Embodiment 39 is the homogeneous aqueous solution of embodiment 35, wherein the transition metal complex is a Cu complex, preferably a Cu(I) complex. Embodiment 40 is the homogeneous aqueous solution of embodiment 35, wherein the transition metal complex is an Ag complex, preferably an Ag(I) complex. Embodiment 41 is the homogeneous aqueous solution of any one of embodiments 32 to 40, wherein the leaving group dissociates from the transition metal complex in response to light. Embodiment 42 is the homogeneous aqueous solution of embodiment 41, wherein the leaving group is CN⁻. Embodiment 43 is the homogeneous aqueous solution of embodiment 42, wherein the transition metal complex is ferricyanide (Fe(CN)₆)⁴⁻) or a salt thereof. Embodiment 44 is the homogeneous aqueous solution of any one of embodiments 41 to 43, wherein the light is sunlight or artificial light, or a combination thereof. Embodiment 45 is the homogeneous aqueous solution of embodiment 44, wherein the artificial light is from a xenon lamp, a fluorescent light, an LED light, an incandescent light, an ultraviolet (UV) light, or any combination thereof. Embodiment 46 is the homogeneous aqueous solution of any one of embodiments 32 to 41, wherein the leaving group dissociates from the transition metal in response to the basic pH of the solution. Embodiment 47 is the homogeneous aqueous solution of embodiment 46, wherein the leaving group is a halide. Embodiment 48 is the homogeneous aqueous solution of embodiment 47, wherein the halide is fluoride (F⁻), chloride (Cl⁻), bromide (Br⁻), iodide (I⁻), or astatide (At⁻), preferably Cl⁻. Embodiment 49 is the homogeneous aqueous solution of any one of embodiments 32 to 48, wherein the molar ratio of formaldehyde to base is equal to or less than 2:1, preferably equal to or less than 1.5:1, more preferably equal to or less than 1.2:1, even more preferably from 0.5:1 to 1.5:1, or most preferably from 1:1 to 1.3:1. Embodiment 50 is the homogeneous aqueous solution of embodiments 32 to 49, wherein the formaldehyde is para-formaldehyde, hydrated formaldehyde, or a combination thereof. Embodiment 51 is the homogeneous aqueous solution of any one of embodiments 32 to 50, wherein the base is NaOH. Embodiment 52 is the homogeneous aqueous solution of any one of embodiments 32 to 51, wherein the homogeneous aqueous solution has a pH from 8 to 14, preferably 10 to 14, and most preferably 12 to 14. Embodiment 53 is the homogeneous aqueous solution of embodiments 32 to 52, wherein the formaldehyde and the transition metal complex are fully solubilized in the homogeneous aqueous solution.

Embodiment 54 is a system for producing hydrogen (H₂) gas and formate or a salt thereof from formaldehyde. The system of embodiment 54 includes a container comprising the homogeneous aqueous solution of any one of embodiments 32 to 53; and optionally, a light source for illuminating the aqueous solution. Embodiment 55 is the system of embodiment 54, wherein the light source is sunlight or an artificial light source, or a combination thereof. Embodiment 56 is the system of embodiment 55, wherein the artificial light source is a xenon lamp, a florescent light, an LED light, an incandescent light, an ultraviolet (UV) light, or any combination thereof. Embodiment 57 is the system of any one of embodiments 54 to 56, wherein the container comprises a transparent portion. Embodiment 58 is the system of any one of embodiments 54 to 57, wherein the container comprises an opaque portion. Embodiment 59 is the system of any one of embodiments 54 to 58, wherein the system does not include an external bias to produce hydrogen or formate or a salt thereof.

The following includes definitions of various terms and phrases used throughout this specification.

The terms “homogeneous mixture” or “homogenous aqueous solution” are defined as a reaction equilibrium in which a single phase exists. For example, and in one non-limiting aspect of the present invention, the formaldehyde and the transition metal complex present in the homogenous aqueous solution of the present invention can both be substantially solubilized or fully solubilized in the aqueous solution.

The term “heterogeneous mixture” is defined as a reaction mixture that contains two or more phases. For example, and in one non-limiting aspect of the present invention, a heterogeneous mixture can be obtained by adding to a homogenous aqueous solution a catalyst that is suspended in the aqueous solution and is capable of catalyzing the hydrogenation of the produced formate to formaldehyde. In this example, two phases exist: (1) the homogenous aqueous solution having formaldehyde (e.g., para-formaldehyde) and the transition metal complex solubilized therein; and (2) the catalyst that is capable of catalyzing the hydrogenation of formate with the water present in the aqueous solution to produce formaldehyde, wherein this catalyst is suspended in the aqueous solution.

The term “heterogeneous catalyst” is a catalyst that is not solubilized in the aqueous solution of the present invention, such that two or more phases are present: (1) the aqueous solution; and (2) the heterogeneous catalyst.

The term “formaldehyde” as used herein includes gaseous, liquid and solid forms of formaldehyde. “Formaldehyde” includes its aldehyde form (CH₂O), its hydrated form (methanediol), and its para-formaldehyde form of

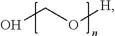

where n can be up to 100.

The term “formate” includes the formate anion (HCOO⁻) or salt forms thereof (e.g., (HCOO⁻)X, where X is a metal cation (e.g., Li⁺, Na⁺, K⁺, Rb⁺, Cs⁺, or Fr⁺).

The terms “about” or “approximately” are defined as being close to as understood by one of ordinary skill in the art. In one non-limiting embodiment, the terms are defined to be within 10%, preferably within 5%, more preferably within 1%, and most preferably within 0.5%.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising,” “including,” “containing,” or “having” in the claims, or the specification, may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The methods, compositions and systems of the present invention can “comprise,” “consist essentially of,” or “consist of” particular ingredients, components, compositions, etc. disclosed throughout the specification. With respect to the transitional phase “consisting essentially of,” in one non-limiting aspect, a basic and novel characteristic of the methods, compositions, and systems of the present invention is the generation of hydrogen and formate from formaldehyde and the further hydrogenation of formate to formaldehyde. Both of these reactions can occur in the same basic aqueous solution.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

Advantages of the present invention may become apparent to those skilled in the art with the benefit of the following detailed description and upon reference to the accompanying drawings.

FIGS. 1A-D are schematics for the production of hydrogen from formaldehyde of the present invention.

FIG. 2 shows graphs of formaldehyde production from sodium formate after 5 hours of illumination with various metal oxide photocatalysts.

FIG. 3 shows graphs of formaldehyde and oxygen production from sodium formate after 5 hours of illumination with a Bi₂WO₆ photocatalyst of the present invention.

FIG. 4 shows graphs of hydrogen and oxygen production from sodium formate after 5 hours of illumination with a Bi₂WO₆ photocatalyst and a Na₄Fe(CN)₆ catalyst of the present invention.

FIG. 5 shows graphs of hydrogen generation with both Bi₂WO₆ and Na₄Fe(CN)₆, only Bi₂WO₆ and with only Na₄Fe(CN)₆ as well as the formaldehyde generated by Bi₂WO₆.

While the invention is susceptible to various modifications and alternative forms, specific embodiments thereof are shown by way of example in the drawings and may herein be described in detail. The drawings may not be to scale.

### DETAILED DESCRIPTION OF THE INVENTION

A discovery has been made that provides for an efficient and scalable process for producing hydrogen gas from formaldehyde. The process includes subjecting an aqueous basic solution having a transition metal complex having a coordination bond between a transition metal and a leaving group that dissociates from the transition metal complex in response to light (e.g., iron containing photocatalyst) and/or the basic pH of the solution, formaldehyde (e.g., methanediol or para-formaldehyde or a combination thereof), and a base to light (e.g., natural or artificial light or a combination thereof), and producing hydrogen gas and formate or a salt thereof from the formaldehyde. The formate or salt thereof can be hydrogenated by hydrogen contained in the water to produce formaldehyde, thereby renewing the hydrogen source. As illustrated in non-limiting embodiments in the examples, this process can have 1) large turn-over numbers, amd 2) be operated at relatively low temperatures (e.g., room temperatures such as 15° C. to 30° C., or more preferable 20° C. to 30° C., or even more preferably from 20° C. to 25° C.) and under a variety of conditions, thereby allowing for the efficient and scalable production of hydrogen gas. In certain instances, production of unwanted by-products such as carbon dioxide can be avoided. The method is efficient as the hydrogen evolution is in a homogeneous fashion. Reaction scheme A provides a non-limiting illustration of a method of the present invention using a transition metal complex (e.g., sodium ferrocyanide) as the catalyst for the homogeneous production of hydrogen and formate salt, and a heterogeneous catalyst (e.g., bismuth tungstate) for the subsequent hydrogenation of the formate salt to produce the hydrated form of formaldehyde (methanediol) and oxygen. The formaldehyde can then be dehydrogenated to form formate ion to continue the cycle, with the net result being the production of hydrogen and oxygen.

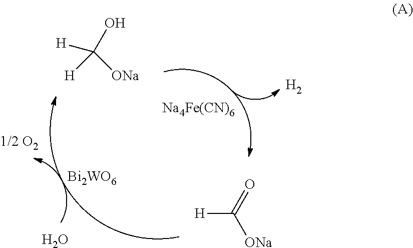

These and other non-limiting aspects of the present invention are discussed in further detail in the following sections.

### A. Transition Metal Complex Catalyst

In some instances, a transition metal complex having a coordination bond between the transition metal and a leaving group acts as a catalyst for the production of formate and H₂ from formaldehyde. The transition metal complex can undergo a reversible dissociation reaction of at least one leaving group. Without wishing to be bound by theory, it is believed that the dissociation of at least one leaving group can produce a transient electrophilic species. A non-limiting example of a transition metal complex catalyst undergoing a dissociation reaction is shown in equation (10) below:

[M^{a}(Zₙ)^{b}(Lo)^{x}]^{y}⇄[M^{a}(Zₙ)^{b}]^{y}+(Lₒ)^{x} (10)

where M is a transition metal having a charge a, Z is one or more ligands bonded to the metal with a total charge of b, L is one or more leaving group with total charge of x, and a is a positive integer from 0 to 6, preferably 0 to 3, b is an negative integer from 0 to −5, x is a negative integer from −1 to −2, y is the total charge of the transition metal complex, and n and o are the atomic ratio relative to M, where n ranges from 0 to 6 and o ranges from 1 to 3. In some instances y is 0, −1, −2, −3, −4, −5, or −6.

The transition metal complex can react with nucleophiles in the reaction mixture, for example, hydroxide ion as shown in equation (11) below.

[(M)^{a}(Zₙ)^{b}(Lₒ)^{x}]^{y}+(OH⁻)ₚ⇄[(M)^{a}(Zₙ)^{b}(OH⁻)ₚ]^{y} (11)

where M is a transition metal having a charge a, Z is one or more ligands bonded to the metal with a total charge of b, L is one or more leaving group with total charge of x, and a is a positive integer from 0 to 6, preferably 0 to 3, b is an negative integer from 0 to −5, x is a negative integer from −1 to −2, y is the total charge of the transition metal complex, and n, o, and p are the atomic ratio relative to M, where n is ranges from 0 to 6, o ranges from 1 to 3, and p ranges from 0 to 1. In some instances y is 0, −1, −2, −3, −4, −5, or −6.

Without wishing to be bound by theory, it is believed that the [(M)^{a}(Zₙ)^{b}(OH⁻)ₚ]^{y} species can react with small organic molecules (e.g., formaldehyde in either intact or hydrated form), followed by reductive elimination of hydrogen and consequent formation of the formate anion. Alternatively, the partly deprotonated form of methanediol (CH₂(OH)₂), as obtained from the attack of hydroxide ion to p-formaldehyde, may also directly coordinate to the [(M)^{a}(Zₙ)^{b}(OH)ₚ]^{y} intermediate to form the same species.

In some instances, the transition metal in the transition metal complex catalyst can be, for example, iron (Fe), ruthenium (Ru), iridium (Ir), or silver (Ag). Preferably, the transition metal is Fe(II), Ru(III), Ir(III), Cu(I), or Ag(I). In some instances, the leaving group (L) can be from two general categories: (1) leaving groups that dissociate from the transition metal complex in response to light and (2), leaving groups that dissociate from the transition metal complex in response to the basic pH of the solution. The former category of leaving groups can include, for example, CN⁻. The latter category can include, for example, halides, including fluoride (F⁻), chloride (Cl⁻), bromide (Br⁻), iodide (I⁻), or astatide (At⁻). Ligand Z can be the same or different than leaving group L. In some embodiments, Z can be an inorganic ligand, an organic ligand or both. Non-limiting examples of organic groups include aromatic groups, a cyano group, a substituted cyano group, an acetate group, a thiocyanate group, an aminidate group, a nitrate group, or combinations thereof. Non-limiting examples of inorganic groups include a halide, phosphate, or both. In some complexes Z is not necessary (e.g., when M has a charge of +1).

In some instances, the transition metal complex contains iron and has a cyano (CN⁻) leaving group. The iron containing catalyst can be a saturated 18-electron complex with Fe(II) in an octahedral, strong ligand-field. The iron containing catalyst can undergo a reversible dissociation reaction of at least one leaving group upon irradiation with visible light. Without wishing to be bound by theory, it is believed that the dissociation of at least one leaving group can produce a transient penta-coordinated 16-electron species isolobal with an organic carbocation. Such an electrophilic species can react with nucleophiles. A non-limiting example of such an iron(II) complex is ferrocyanide ([Fe(CN)₆]⁴⁻). In this instance, leaving group CN and ligand Z are the same group. Ferrocyanide is available from many commercial manufacturers, for example, Sigma Aldrich® (USA), as sodium ferrocyanide decahydrate ([(CN)₆Fe]Na₄(H₂O)₁₀). A non-limiting example of an iron containing catalyst, ferrocyanide, undergoing a reversible dissociation reaction is shown in equation (12) below.

[Fe(CN)₆]⁴⇄[Fe(CN)₅]³⁻+CN⁻ (12)

The iron containing catalyst can react with nucleophiles in the reaction mixture, for example, hydroxide ion as shown in equation (13) below.

[Fe(CN)₅]³⁻+OH⁻⇄[Fe(CN)₅(OH)]⁴⁻ (13)

Without wishing to be bound by theory, it is believed that the [Fe(CN)₅(OH)]⁴⁻ species is responsible for the reaction with small organic molecules (e.g., formaldehyde in either intact or hydrated form), followed by reductive elimination of hydrogen and consequent formation of the formate anion as shown in the reaction pathway (B) below. Alternatively, the partly deprotonated form of methanediol (CH₂(OH)₂), as obtained from the attack of hydroxide ion to p-formaldehyde, may also directly coordinate to the 16-electron [Fe(CN)₅]³⁻ intermediate to form the same species as shown in reaction pathway (B) below, where Z is CN, a is +2, n is 5, and b is −5.

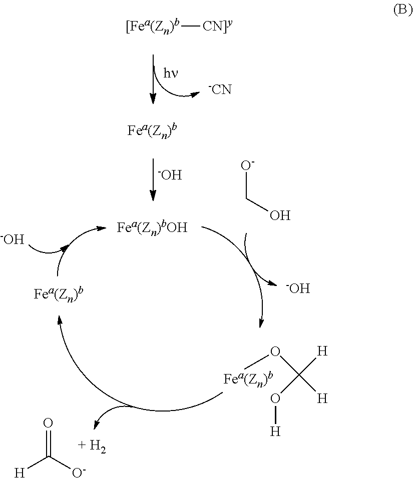

A non-limiting example of a transition metal complex undergoing a reversible dissociation reaction under basic pH is shown in reaction pathway (C) below. In a preferred embodiment, Z and L are halides.

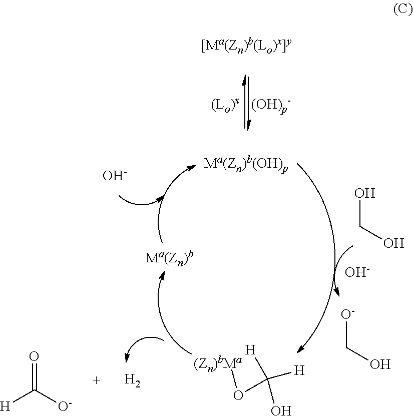

where M is a transition metal having a charge a, Z is a ligand bonded to the metal with a charge of b, L is a leaving group with a charge of x, and a is a positive integer from 0 to 6, preferably 0 to 3, b is an negative integer from 0 to −5, x is a negative integer from −1 to −2, y is the total charge of the transition metal complex, and n, o, and p are the atomic ratio relative to M, where n is 0 to 6, o is 1 to 3, p is 0 to 1, and y is 0, −1, −2, −3, −4, −5, or −6.

### B. Hydrogenation Photocatalyst

The hydrogenation catalyst can be any catalyst capable of catalyzing a hydrogenation reaction as shown in reaction Scheme (D).

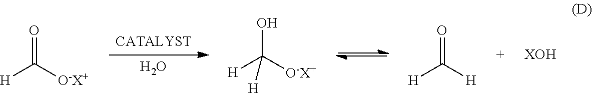

where X is a metal cation.

The hydrogen source can be water and/or hydrogen evolved in the reaction of the formaldehyde with the transition metal complex catalyst. In some embodiments, the hydrogenation catalyst is a heterogeneous catalyst capable of catalyzing the hydrogenation of formate a salt thereof with hydrogen reaction. The catalyst can be a heterogeneous metal oxide photocatalyst. The photocatalyst can include active metals such as bismuth (Bi), tungsten (W), chromium (Cr) vanadium (V), lantheum (La), cobalt (Co), copper (Cu) gold (Au) or any combination thereof. Non-limiting examples of the photocatalyst include Bi₂WO₆, BiVO₄, LaCoO₃, CuWO₄, BiCu₂VO₆, Au/TiO₂, Cr₂WO₆, or combinations thereof. The metal oxide photocatalyst can be obtained from a commercial source (e.g., Sigma-Aldrich®, USA) or prepared from metal precursors such as metal nitrates.

### C. Reactants and Medium for Production of Hydrogen, Formate, Formaldehyde

1. Reactants

The reactants in the step of producing formate and H₂ can include formaldehyde, paraformaldehyde, or other organic molecules that release formaldehyde in aqueous solution. Formaldehyde can be formaldehyde, aqueous formaldehyde solutions (for example 37% in water), para-formaldehyde, or combinations thereof. para-Formaldehyde is the polymerization of formaldehyde with a typical degree of polymerization of 1 to up to 100 units. Aqueous formaldehyde (methanediol) and para-formaldehyde are available from many commercial manufacturers, for example, Sigma Aldrich® (USA). In addition, reactants can include small organic molecules with a terminal aldehyde (RHCO), where R is H or an alkyl group having 1 to 3 carbons. The basic reagent can include a metal hydroxide (MOH or M(OH)₂), where M is a alkali or alkaline earth metal. Non-limiting examples of alkali or alkaline earth metals include lithium, sodium, potassium, magnesium, calcium, and barium. In a preferred embodiment, the base is sodium hydroxide (NaOH). The molar ratio of small organic molecule (e.g., formaldehyde) to base is equal to or less than 2:1, 1.9:1, 1.8:1, 1.7:1, 1.6:1, 1.5:1, 1.2:1, 1.1:1, 1:1, 0.5:1 or any range there between.

2. Medium

The production of hydrogen and formate from formaldehyde, and the production of formaldehyde from formate can be performed in any type of medium that can solubilize the transition metal complex catalyst and reagents. In a preferred embodiment, the medium is water. Non-limiting examples of water include de-ionized water, salt water, river water, canal water, city canal water, mixtures thereof, or the like.

D. Hydrogen and Oxygen Production from Aqueous Formaldehyde

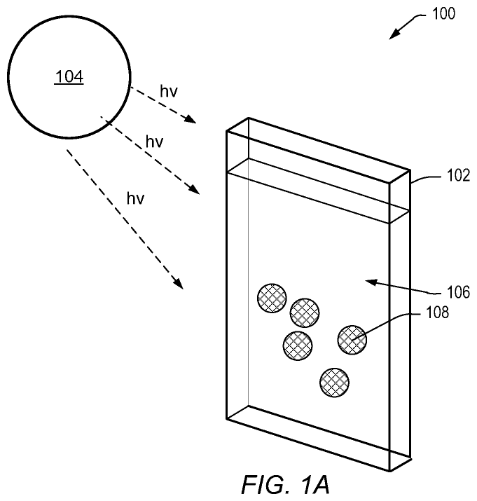

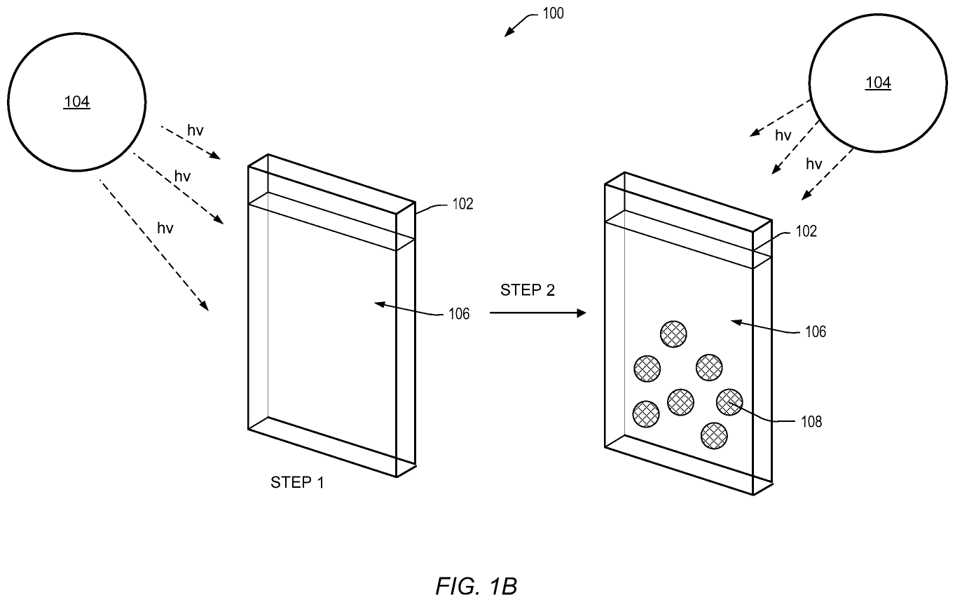

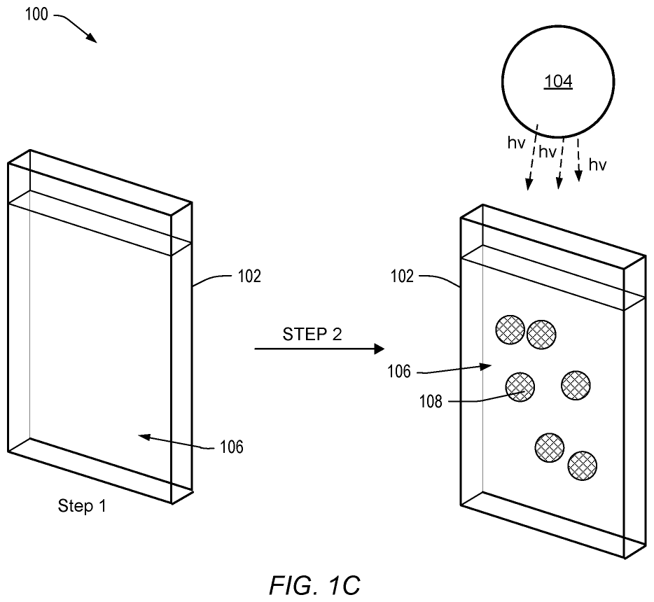

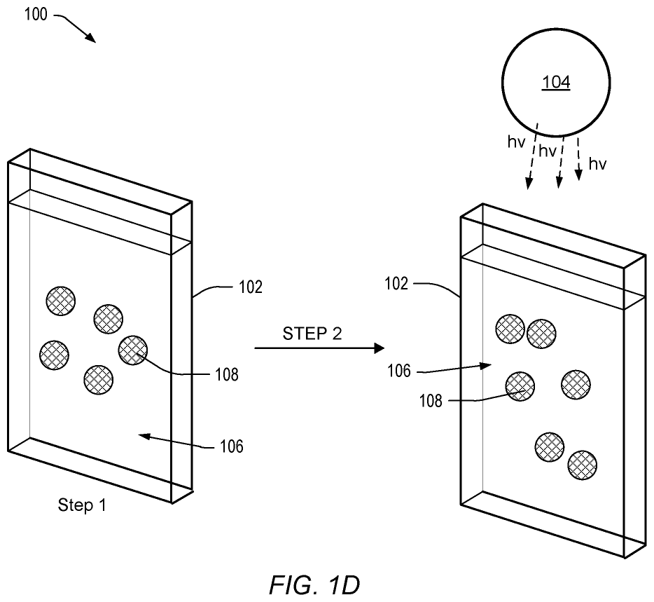

Hydrogen and formate can be produced by irradiating, with light, an aqueous composition having a basic pH, formaldehyde, and a homogeneous transition metal complex catalyst and a heterogeneous metal oxide photocatalyst. In preferred instances, the transition metal complex catalyst and the formaldehyde are partially or fully solubilized within the aqueous composition. FIG. 1A is a schematic of an embodiment of a reaction system 100 for producing hydrogen and formate from formaldehyde. System 100 is particularly suited to methods that use a transition metal complex catalyst having a leaving group that dissociates from the transition metal complex in response to light. System 100 includes container 102, light source 104, and aqueous mixture 106. Container 102 can be transparent, translucent, or even opaque such as those that can magnify light (e.g., opaque container having a pinhole(s) or those that include a light source within the container). The aqueous mixture 106 includes the homogeneous mixture of aqueous formaldehyde (methanediol), a transition metal complex catalyst, and a base described throughout the specification and the heterogeneous metal oxide catalyst 108. Light source 104 can be natural sunlight or an artificial light source such as light from a xenon lamp, a fluorescent light, a light emitting diode (LED), an incandescent light, an ultraviolet (UV) light, or any combination thereof. In certain instances, a combination of natural and artificial light can be used. The transition metal complex catalyst can be used to catalyze the production of formate and hydrogen from the formaldehyde as shown in reaction pathways (A) (B) and (C) above and the heterogeneous metal oxide photocatalyst 108 can be used to catalyst the production of formaldehyde from formate or a salt thereof and water or hydrogen gas as shown in reaction pathway (A) above. When the aqueous mixture 106 is exposed the light source 104, H₂ (gas) and formate are produced. The produced formate is subsequently hydrogenated with hydrogen from the water to produce formaldehyde, thereby providing a renewable source of hydrogen and oxygen and formaldehyde. Notably, formate, hydrogen, formaldehyde and oxygen are produced only when the solution containing the catalyst is exposed to light. No formate, hydrogen, formaldehyde, or oxygen is produced when aqueous formaldehyde and sodium hydroxide solution alone are exposed to light. Thus, it should be understood that you can either illuminate and then add the catalyst or add the catalyst and then illuminate the solution. In some embodiments, the method is performed in two steps as shown in FIG. 1B. In step 1, production of formate and hydrogen can occur as described above. In step 2, the heterogeneous photocatalyst can be added and the container can be illuminated to catalyze the hydrogenation of the produced formate ion.

System 100 can also be used in embodiment when the leaving group dissociates in response to the basic pH of the solution (for example, as shown in pathway (C) above). System 100 is particularly suited to methods that use a transition metal complex catalyst having a leaving group that dissociates from the transition metal complex in response to pH. In such a system, light source 104 is not necessary to promote the production of formate ion and hydrogen. A light source can be used to promote the hydrogenation of formate ion to formaldehyde. FIGS. 1C and 1D depict schematics of the production of hydrogen, formate, formaldehyde and oxygen. In step 1, the reaction to produce hydrogen and formate can be performed as described above with response to pH. This reaction can be monitored, and in step 2, when a sufficient amount of formate ion is produced, the heterogeneous metal oxide photocatalyst can be added to the solution. Light can be provided to the solution to catalyze the hydrogenation of formate with water to produce formaldehyde as shown in FIG. 1C to continue the cycle. As shown in FIG. 1D, the metal oxide catalyst 108 can be added in step 1, but does not catalyze the hydrogenation reaction until light is applied in step 2.

When equimolar solutions of p-formaldehyde and sodium hydroxide are combined, a slow Cannizzaro's disproportionation to MeOH and (HCOO)Na can occur as shown in equation (14) below. The addition of a catalytic amount of the transition metal catalyst containing does not appear to inhibit this disproportionation.

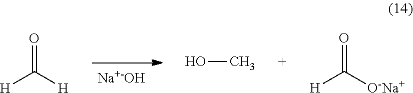

The production of formate (e.g., sodium formate) can be as illustrated in the reaction pathways (A), (B), and (C) above and equation (15) below.

CH₂O(l)+NaOH(aq)→H₂(g)+HCOONa(aq) Δ [symbol] =−91 kJ/mol (15)

Without wishing to be bound by the theory, the production of hydrogen is in the homogeneous phase of the aqueous mixture. The spent transition metal complex (e.g., (M)^{a}(Zₙ)^{b}) can be precipitate or be precipitated from the solution by addition of acid to increase the pH of the solution. The resulting precipitate can be removed, or substantially removed, through known solid/liquid filtration methods (e.g., centrifugation, filtration, gravity settling, etc.). In some embodiments, the transition metal complex is not removed or is partially removed from the solution. The formate (or formic acid), which is also dissolved in the solution, can then be used as a carbon source for production of formaldehyde or the hydrated form of formaldehyde (methanediol).

In some embodiments, the sodium formate can be used as a starting material to generate oxygen and formaldehyde in situ using the heterogeneous metal oxide photocatalyst. For example, a solution of sodium formate, homogeneous catalyst, heterogeneous metal oxide photocatalyst can be irradiated to produce formaldehyde and oxygen. The formaldehyde (methanediol) formed in situ can then be reduced to form hydrogen and formate by contact with the homogenous catalyst in response to light or change in base to continue the cycle.

Notably, no carbon dioxide is formed during the production of formate and hydrogen and the formaldehyde starting material is regenerated through the hydrogenation of formate reaction. Thus, the process can be considered a cyclic “green” process. Furthermore, system 100 does not require the use of an external bias or voltage source, although one can be used if so desired. Further, the efficiency of system 100 allows for one to use formaldehyde as a hydrogen storage agent and formate as a carbon source.

### EXAMPLES

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

### Example 1

### (Materials and Testing Procedures For Production of Hydrogen from Formaldehyde)

Materials. All materials were purchased from Sigma-Aldrich® (USA). Chemicals were used without further purification. If not specifically mentioned, all reactions were carried out in distilled water without degassing or other modifications.

Product Analysis. H₂, CO₂, CO and O₂ gas identification and detection was carried out with an Agilent 7820A GC equipped with a thermal conductivity detector (TCD), using an Agilent GS-CarbonPlot column (for CO₂) or Agilent HP-Molesieve column (for all other gasses).

Synthesis of Bi₂WO₆. Sodium tungstate dihydrate (2.5 mmoles) was dissolved in water (30 mL) to form a solution. This solution was added dropwise to aqueous bismuth nitrate (5 mmoles in 20 mL water) while stirring. After addition of the sodium tungstate, the solution was stirred for a further 10 minutes and sonicated for 20 minutes. This solution was poured into a 100 mL pressure tube and water (30 mL) was added. The solution was heated to 160° C. for 20 hours, after which the yellow-white precipitate was collected by centrifuge and washed with water (3×50 mL). The powder obtained was then dried in an oven at 80° C. overnight.

### Example 2

### (Generation of Formaldehyde from Sodium Formate with Various Catalysts)

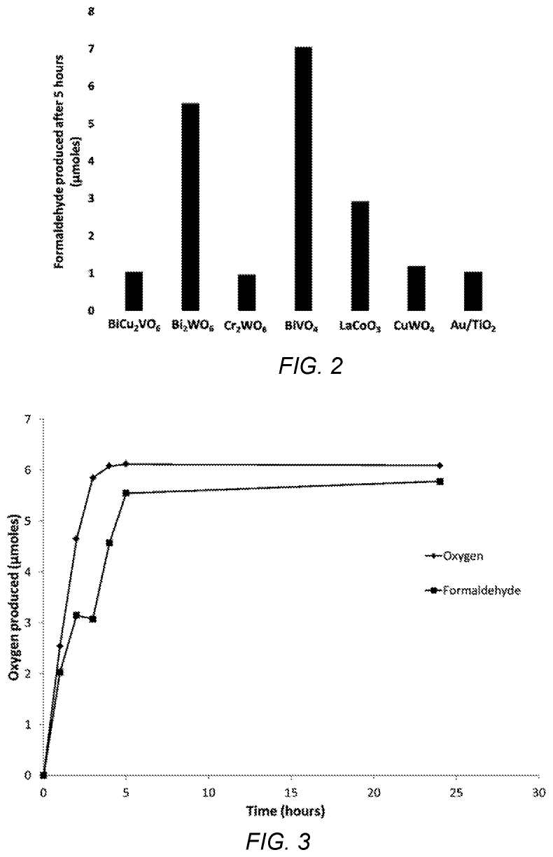

Sodium formate (15 mL, 7.4 mmol of sodium formate) in H₂O and the heterogeneous metal oxide photocatalyst (140 μmol) were mixed together. The reaction mixture was illuminated with a 300 W Xe arc-lamp for 5 hours and the production of oxygen and formaldehyde was monitored. FIG. 2 are graphs of formation of formaldehyde for the catalysts tested. As shown in FIG. 2, formaldehyde was produced in case, which indicated that formate was consumed in the reaction process.

### Example 3

### (Generation of Formaldehyde from Sodium Formate with Bismuth Tungstate)

Sodium formate (15 mL, 7.4 mmol of sodium formate) in H₂O was mixed with the heterogeneous metal oxide photocatalyst (Bi₂WO₆, 140 μmol). The reaction mixture was illuminated with a 300 W Xe arc-lamp for 24 hours and the production of oxygen and formaldehyde was monitored. FIG. 3 are graphs of formation of formaldehyde (bottom line) and oxygen (top line). As shown in FIG. 3, formaldehyde was produced in case, which indicated that formate and water was consumed in the reaction process. After 5 hours, both the oxygen and formaldehyde levels had reached the maximum. The overproduction of oxygen was attributed to an increase in acidity in the solution as shown in the equations 16 and 17 below.

H₂O→1/2O₂+2H⁺+2e⁻ (16)

2H⁺+2C+HCOONa→H₂C(OH)(ONa) (17)

Another reason for the increased oxygen content was further reduction of formaldehyde to methanol, although this was below detection limit of the gas chromatograph. The formic acid to methanol route through formaldehyde is a very well-known pathway for CO₂ reduction, and without wishing to be bound by theory, it is believed this pathway could be responsible for the increased O₂ levels compared to formaldehyde.

### Example 4

### (Generation of Formaldehyde from Sodium Formate with Bismuth Tungstate)

Water (15 mL) was placed in a crimp-top vial. To this, sodium formate (7.4 mmoles) was added along with sodium hydroxide (13 mmoles). Once these had dissolved, BiWO₆ (140 μmoles) and Na₄Fe(CN)₆ (100 μmoles) were added with vigorous stirring, followed by sealing the vial and placing in front of the light source. The gas production was monitored for up to 24 hours by GC and formaldehyde was monitored by a colorimetric test.

Formaldehyde determination. To a solution of ammonium acetate (15.4 g) in water (50 mL), acetyl acetone (0.2 mL) and glacial acetic acid (0.3 mL) were added while stirring. This was further diluted with water (49.5 mL) and stored in a refrigerator for up to 3 days. To determine the formaldehyde concentration, the sample (2 mL) was mixed with an equal amount of the acetyl acetone solution (2 mL) and heated to 60° C. for 10 minutes. After cooling for 10 minutes, the absorbance of the solution was measured at 412 nm and compared to a calibration curve.

### Example 5

### (Generation of Formaldehyde from Sodium Formate in the Presence of Base and Transition Metal Complex Catalyst)

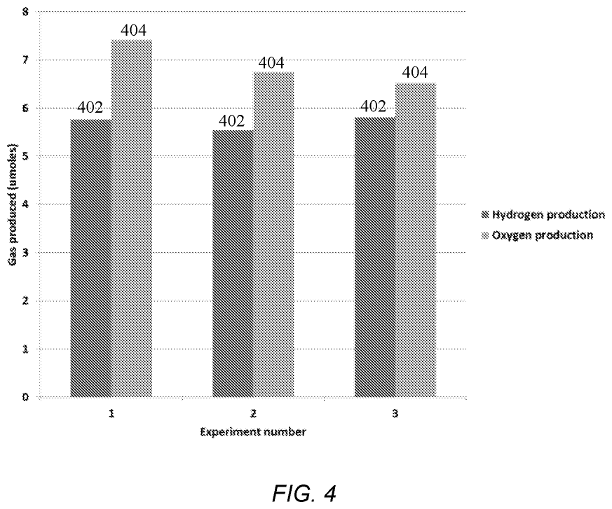

Sodium formate (15 mL, 7.4 mmol of sodium formate), sodium hydroxide (13 mmoles), homogeneous photocatalyst (Na₄Fe(CN)₆, 100 μmoles) and the heterogeneous metal oxide photocatalyst (Bi₂O₆, 140 μmol) was mixed together. The reaction mixture was illuminated with a 300 W Xe arc-lamp for 24 hours and the production of hydrogen and oxygen was monitored. FIG. 4 are graphs of formation of hydrogen (bars 402) and oxygen (bars 404) for three trials. As shown in FIG. 4, oxygen and hydrogen were both produced in each trial, which indicated that carbon mediated water splitting was performed (i.e., oxygen must be produced in addition to hydrogen). For a pure water splitting system, a 2:1 hydrogen:oxygen ratio would be expected. In this example, however, more oxygen was produced. Without wishing to be bound by theory, it was believed that some oxygen was formed from the Cannizzaro reaction discussed above which produces both methanol and formate from formaldehyde in base, and the reasons shown Example 3.

### Example 6

### (Comparative Experiments)

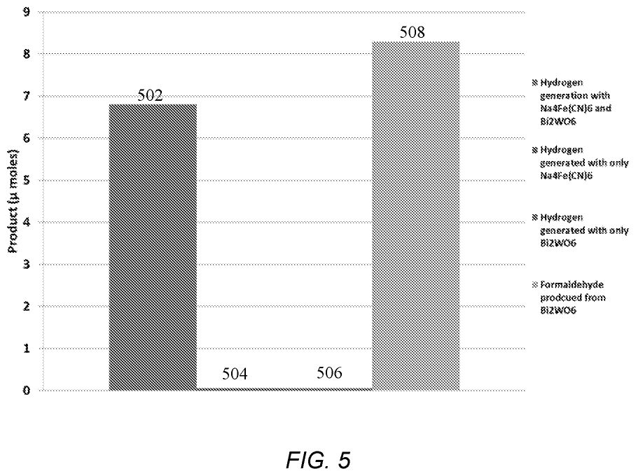

To confirm that the hydrogen was from the dehydrogenation of the photo-generated formaldehyde, Example 4 was repeated two more times while neglecting one of the two catalysts each time (FIG. 5). FIG. 5 shows graphs of product produced and catalyst used. Bar 502 depicts hydrogen generated when both Na₄Fe(CN)₆ and Bi₂O₆ are present, bar 504 depicts hydrogen generated when only Na₄Fe(CN)₆ is present (Bi₂O₆ is not used), bar 506 depicts hydrogen generated when only Bi₂O₆ is present (Na₄Fe(CN)₆ is not used), and bar 508 depicts formaldehyde produced when only Bi₂O₆ is present (Na₄Fe(CN)₆ is not used). When one of the two catalysts was missing from the reaction mixture, no hydrogen was detected, confirming the hydrogen was coming from the dehydrogenation of the formaldehyde produced from the reduction of formate. Without wishing to be bound by theory, it is believed that this could lead to a water splitting cycle which can store hydrogen until needed.

1. A method of producing hydrogen, the method comprising:
(a) combining an aqueous base, formaldehyde, and a transition metal complex having a coordination bond between a transition metal and a leaving group to form a homogeneous aqueous solution having a basic pH, wherein the leaving group dissociates from the transition metal complex in response to light and/or the basic pH of the solution;
(b) producing hydrogen (H₂) gas and formate or a salt thereof from the formaldehyde present in the homogeneous aqueous solution; and
(c) hydrogenating the formate or a salt thereof to produce formaldehyde.

2. The method of claim 1, wherein the formate or salt thereof is hydrogenated with hydrogen obtained from water comprised in the homogenous aqueous solution.

3. The method of claim 2, wherein a heterogeneous catalyst catalyzes the hydrogenation of formate or a salt thereof with water reaction.

4. The method of claim 3, wherein the heterogeneous catalyst is a metal oxide photocatalyst selected from Bi₂WO₆, BiVO₄, LaCoO₃, CuWO₄, BiCu₂VO₆, Au/TiO₂, Cr₂WO₆, or combinations thereof.

5. The method of claim 1, wherein the formaldehyde produced from step (c) is recycled/used in steps (a) and/or (b).

6. The method of claim 1, wherein oxygen gas (O₂) is produced in step (c).

7. The method of claim 1, wherein steps (a), (b), and/or (c) are each performed at a temperature from greater than 0° C. to less than 50° C.

8. The method of claim 1, wherein the transition metal is iron (Fe), ruthenium (Ru), iridium (Ir), copper (Cu), or silver (Ag).

9. The method of claim 1, wherein the leaving group dissociates from the transition metal complex in response to light.

10. The method of claim 9, wherein the transition metal complex is ferricyanide (Fe(CN)6)⁴⁻) or a salt thereof.

11. The method of claim 1, wherein the leaving group dissociates from the transition metal in response to the basic pH of the solution.

12. The method of claim 11, wherein the leaving group is a halide.

13. The method of claim 1, wherein the formaldehyde is para-formaldehyde, hydrated formaldehyde, or a combination thereof and the base is NaOH.

14. An aqueous solution having a basic pH, the composition comprising a solution of an aqueous base, formaldehyde, formate, a heterogeneous catalyst, and a homogeneous catalyst comprising a transition metal complex having a coordination bond between a transition metal and a leaving group.

15. (canceled)

16. The aqueous solution of claim 14, wherein the heterogeneous catalyst is a photocatalyst selected from Bi₂WO₆, BiVO₄, LaCoO₃, CuWO₄, BiCu₂VO₆, Au/TiO₂, Cr₂WO₆, or any combination thereof.

17. The aqueous solution of claim 14, wherein the transition metal complex metal is iron (Fe), ruthenium (Ru), iridium (Ir), copper (Cu), or silver (Ag).

18. The aqueous solution of claim 14, wherein the leaving group dissociates from the transition metal complex in response to light or the basic pH of the solution.

19. A system for producing hydrogen (H₂) gas and formate or a salt thereof from formaldehyde, the system comprising:
(a) a container comprising the aqueous solution of claim 14; and
(b) optionally, a light source for illuminating the aqueous solution.

20. The system of claim 19, wherein the system does not include an external bias to produce hydrogen or formate or a salt thereof

21. The method of claim 7, wherein the temperature is from 10° C. to 40° C.
